(19) United States
(12) Patent Application Publication
(10) Pub. No.: US 2021/0015830 A1
(43) Pub. Date: Jan. 21, 2021

(54) COMPOSITION FOR INHIBITING REACTIVE OXYGEN SPECIES PRODUCTION

(71) Applicant: SUNTORY HOLDINGS LIMITED, Osaka-shi, Osaka (JP)

(72) Inventors: Yuki Yagita, Soraku-gun (JP); Ayuta Funaki, Soraku-gun (JP); Naoki Kasajima, Soraku-gun (JP); Yoshiko Ono, Soraku-gun (JP); Daisuke Takemoto, Soraku-gun (JP)

(73) Assignee: SUNTORY HOLDINGS LIMITED, Osaka-shi, Osaka (JP)

(21) Appl. No.: 16/957,490

(22) PCT Filed: Dec. 26, 2018
(86) PCT No.: PCT/JP2018/047880

§ 371 Date: Jun. 24, 2020

(30) Foreign Application Priority Data
Dec. 28, 2017 (JP) 2017-253319

(51) Int. Cl. A61K 31/56; A23L 33/10; A61P 25/28; A61P 39/06

(52) U.S. Cl. CPC A61K 31/56; A23L 33/10; A61K 9/0053; A61P 39/06; A61P 25/28

(57) ABSTRACT

The present invention relates to a composition for inhibiting reactive oxygen species production, containing triterpenic acid having an acetyl group and/or a salt thereof as an active ingredient.

### TECHNICAL FIELD

The present invention relates to a composition for inhibiting reactive oxygen species production. The present invention also relates to a method for inhibiting reactive oxygen species production and the use of triterpenic acid having an acetyl group and/or a salt thereof for inhibiting reactive oxygen species production. The present invention also relates to a composition for inhibiting or ameliorating a decline in brain function, a method for inhibiting or ameliorating a decline in brain function, and the use of triterpenic acid having an acetyl group and/or a salt thereof for inhibiting or ameliorating a decline in brain function.

### BACKGROUND ART

Oxidative stress refers to a state in which a living body is inclined toward an oxidized state due to increased reactive oxygen species production, decreased reactive oxygen species removability, and the like. Biomolecules such as proteins, DNAs, and lipids are oxidized due to oxidative stress. The oxidation of such biomolecules is known to be involved in decline in functions of various organs and lead to various diseases (Non Patent Literature 1).

Accordingly, reducing oxidative stress is useful for preventing, ameliorating, or treating various diseases.

Reactive oxygen species in a living body may be produced by electrons leaking from the electron transport system of mitochondria or may be produced through an enzymatic reaction. NADPH oxidase (hereinafter also simply referred to as NOX) is known as an enzyme that produces superoxide, which is one of the reactive oxygen species.

Suppressing NOX to inhibit reactive oxygen species production is known to have the effect of preventing, ameliorating, or treating various conditions and diseases caused by reactive oxygen species. For example, there is a report that inhibiting superoxide production by suppressing NOX inhibits declining cognitive functions (Non Patent Literature 2).

NOX activity is also known to increase with age, and is considered to be involved in accelerated age-related diseases and aging phenomena. Inhibiting NOX activity is considered to be useful for delaying various age-related diseases and aging phenomena.

Methoxyflavone derived from black ginger is also known to be useful as a NOX inhibitor (Patent Literature 1).

On the other hand, superoxide produced in a living body is converted to hydrogen peroxide by superoxide dismutase. Then, hydrogen peroxide is inactivated by being reduced to water by glutathione peroxidase having glutathione as a coenzyme. Glutathione can also directly react with and thus inactivate reactive oxygen species. Accordingly, increasing the glutathione concentration in a body causes reactive oxygen species to be reduced. 3-Acetylursolic acid and 3-acetyloleanolic acid are known to be useful as substances that function to promote glutathione production (Patent Literature 2).

### CITATION LIST

### Patent Literature

Patent Literature 1: JP 2015-227329 A
Patent Literature 2: JP 5466842 B

### Non Patent Literature

Non Patent Literature 1: Neurobiol Aging. 2005 June; 26(6): 857-64
Non Patent Literature 2: PLoS One. 2010 Sep. 7; 5(9): e12606

### SUMMARY OF INVENTION

### Technical Problem

An object of the present invention is to provide a composition for inhibiting reactive oxygen species production, which has a superior action. Another object of the present invention is to provide a composition for inhibiting or ameliorating a decline in brain function, which has a superior action.

### Solution to Problem

Suppressing NOX is considered to be effective as a means for inhibiting reactive oxygen species production and reducing oxidative stress in a body.

The present inventors have found that triterpenic acid having an acetyl group and/or a salt thereof can reduce oxidative stress by inhibiting reactive oxygen species production, and have arrived at the present invention.

That is to say, the present invention relates to (1) to (21) below.

(1) A composition for inhibiting reactive oxygen species production, containing triterpenic acid having an acetyl group and/or a salt thereof as an active ingredient.

(2) The composition for inhibiting reactive oxygen species production according to (1),

wherein the composition inhibits reactive oxygen species production by suppressing NADPH oxidase (NOX).

(3) The composition for inhibiting reactive oxygen species production according to (1) or (2),

wherein the triterpenic acid having an acetyl group and/or the salt thereof is at least one selected from the group consisting of 3-acetylursolic acid, 3-acetyloleanolic acid, and salts thereof.

(4) The composition for inhibiting reactive oxygen species production according to any one of (1) to (3), for use in preventing, ameliorating, or treating a condition or a disease resulting from oxidative stress.

(5) The composition for inhibiting reactive oxygen species production according to any one of (1) to (4), for use in inhibiting or ameliorating a decline in brain function.

(6) The composition for inhibiting reactive oxygen species production according to (5),

wherein the decline in brain function is an age-related decline in brain function.

(7) The composition for inhibiting reactive oxygen species production according to (4) or (5),

wherein the condition or the disease resulting from the oxidative stress or the decline in brain function is at least one condition or disease selected from the group consisting of forgetfulness, decline in cognitive function, decline in memory, decline in judgment, dementia, and Alzheimer's disease.

(8) The composition for inhibiting reactive oxygen species production according to any one of (1) to (7),

wherein the composition is labeled as being intended for preventing, ameliorating, or treating a condition or a disease resulting from oxidative stress or a decline in brain function.

(9) The composition for inhibiting reactive oxygen species production according to any one of (1) to (8),

wherein the composition is an oral composition.

(10) The composition for inhibiting reactive oxygen species production according to (9),

wherein the oral composition is a food or drink, a drug, or a quasi drug.

(11) A method for inhibiting reactive oxygen species production, including administering triterpenic acid having an acetyl group and/or a salt thereof to a subject.

(12) Use of triterpenic acid having an acetyl group and/or a salt thereof for inhibiting reactive oxygen species production.

(13) A composition for inhibiting or ameliorating a decline in brain function, containing triterpenic acid having an acetyl group and/or a salt thereof as an active ingredient.

(14) The composition for inhibiting or ameliorating a decline in brain function according to (13), for use in inhibiting or ameliorating an age-related decline in brain function.

(15) The composition for inhibiting or ameliorating a decline in brain function according to (13) or (14),

wherein the triterpenic acid having an acetyl group and/or the salt thereof is at least one selected from the group consisting of 3-acetylursolic acid, 3-acetyloleanolic acid, and salts thereof.

(16) The composition for inhibiting or ameliorating a decline in brain function according to any one of (13) to (15),

wherein the disease or the condition resulting from the decline in brain function is at least one condition or disease selected from the group consisting of forgetfulness, decline in cognitive function, decline in memory, decline in judgment, dementia, and Alzheimer's disease.

(17) The composition for inhibiting or ameliorating a decline in brain function according to any one of (13) to (16),

wherein the composition is labeled as being intended for preventing, ameliorating, or treating a condition or a disease resulting from a decline in brain function.

(18) The composition for inhibiting or ameliorating a decline in brain function according to any one of (13) to (17),

wherein the composition is an oral composition.

(19) The composition for inhibiting or ameliorating a decline in brain function according to (18),

wherein the oral composition is a food or drink, a drug, or a quasi drug.

(20) A method for inhibiting or ameliorating a decline in brain function, including administering triterpenic acid having an acetyl group and/or a salt thereof to a subject.

(21) Use of triterpenic acid having an acetyl group and/or a salt thereof for inhibiting or ameliorating a decline in brain function.

### Advantageous Effects of Invention

Triterpenic acid having an acetyl group and/or a salt thereof has a remarkable NOX inhibitory activity, moreover, this inhibitory activity is markedly higher than that of triterpenic acid that does not have an acetyl group, and therefore triterpenic acid having an acetyl group and/or a salt thereof has an extremely strong effect of inhibiting reactive oxygen species production. Thus, oxidative stress can be more effectively reduced. Accordingly, the composition for inhibiting reactive oxygen species production of the present invention, which contains triterpenic acid having an acetyl group and/or a salt thereof as an active ingredient, is useful for reducing oxidative stress. Also, the composition for inhibiting reactive oxygen species production of the present invention is useful for inhibiting or ameliorating a decline in brain function.

Also, the composition for inhibiting or ameliorating a decline in brain function of the present invention, which contains triterpenic acid having an acetyl group and/or a salt thereof as an active ingredient, is useful for inhibiting or ameliorating a decline in brain function.

### BRIEF DESCRIPTION OF DRAWINGS

FIG. 1 is a graph showing the latencies (second) in light room of a young control group, an aged control group, and an aged Aronia-received group in an acquisition trial and a retention trial.

FIG. 2 is a graph showing the latencies (second) in light room of an ursolic acid-received group and an acetylursolic acid-received group in an acquisition trial and a retention trial.

### DESCRIPTION OF EMBODIMENTS

The present invention will hereinafter be described in detail. The following embodiments are examples for describing the present invention and are not intended to limit the present invention to those embodiments. The present invention can be carried out in various forms as long as the scope of the present invention is not exceeded.

First, the formulation and the like of the composition for inhibiting reactive oxygen species production of the present invention will now be described.

The composition for inhibiting reactive oxygen species production of the present invention is characterized by containing triterpenic acid having an acetyl group and/or a salt thereof as an active ingredient.

Triterpenic acid having an acetyl group and/or a salt thereof has the effect of inhibiting reactive oxygen species production. Thus, oxidative stress can be effectively reduced. Accordingly, the composition for inhibiting reactive oxygen species production of the present invention, which contains triterpenic acid having an acetyl group and/or a salt thereof as an active ingredient, is useful for reducing oxidative stress.

The composition for inhibiting reactive oxygen species production of the present invention can inhibit reactive oxygen species production by suppressing NOX.

This is due to the effect that triterpenic acid having an acetyl group and/or a salt thereof contained in the composition for inhibiting reactive oxygen species production of the present invention functions as a NOX inhibitor.

Reactive oxygen species cause various types of oxidative stress in a living body, and thus oxidative stress resulting from reactive oxygen species can be suppressed by, for example, taking the composition for inhibiting reactive oxygen species production of the present invention.

Examples of triterpenic acid having an acetyl group and/or a salt thereof contained in the composition for inhibiting reactive oxygen species production of the present invention include acetylursolic acid, acetyloleanolic acid, acetylmaslinic acid, acetylasiatic acid, acetylbetulinic acid, and salts thereof. Among these, preferable is at least one selected from the group consisting of triterpenic acid O-acetylated at 3-position, that is 3-acetylursolic acid, 3-acetyloleanolic acid, and salts thereof. Alternatively, the carboxyl group is not essential as long as an acetyl group is contained, and triterpenic acid having an acetyl group and/or a salt thereof may be at least one selected from the group consisting of ursane-type triterpenes, oleanane-type triterpenes, and salts thereof having an acetyl group.

These compounds have high NOX inhibitory activity.

The salt of triterpenic acid having an acetyl group contained in the composition for inhibiting reactive oxygen species production of the present invention is not limited, and is preferably a salt that can be orally taken. Examples of such salts include salts of alkali metals such as sodium and potassium, salts of alkaline earth metals such as calcium, and ammonium salts.

3-Acetylursolic acid is a compound having a structure represented by formula (1) below.

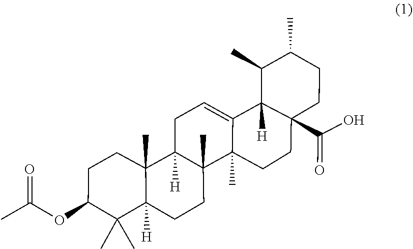

3-Acetyloleanolic acid is a compound having a structure represented by formula (2) below.

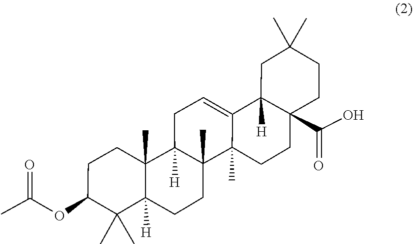

Triterpenic acid having an acetyl group or a salt thereof contained in the composition for inhibiting reactive oxygen species production of the present invention is not limited by what it is derived from or the production method. For example, plant-derived matter extracted from a plant may be used, and a product obtained by synthesis may be used. A commercially available product can be used as well.

When using plant-derived triterpenic acid having an acetyl group and/or a salt thereof, it is preferably derived from at least one plant selected from the group consisting of Aronia, Rubus, Chaenomeles, Malus, Pyrus, Arctostaphylos, Vaccinium, Syzygium, Swertia, Ternstroemia, Ilex, Ficus, Olea, and Eucommia.

These plants contain large amounts of triterpenic acid having an acetyl group and/or a salt thereof, and are thus suitable as raw materials for triterpenic acid having an acetyl group and/or a salt thereof.

When triterpenic acid having an acetyl group and/or a salt thereof contained in the composition for inhibiting reactive oxygen species production is 3-acetylursolic acid, 3-acetyloleanolic acid, or a salt thereof, triterpenic acid having an acetyl group and/or a salt thereof is preferably derived from at least one plant selected from the group consisting of Aronia, Rubus, Chaenomeles, Ternstroemia, Ilex, Ficus, and Eucommia.

Moreover, when 3-acetylursolic acid, 3-acetyloleanolic acid, or a salt thereof is derived from a plant belonging to Aronia, the plant belonging to Aronia is preferably at least one selected from the group consisting of Aronia arbutifolia, Aronia melanocarpa, and Aronia prunifolia.

Aronia melanocarpa is also called chokeberry or black chokeberry. When extracting triterpenic acid having an acetyl group from a plant, it can be obtained by, for example, extracting a raw material containing triterpenic acid having an acetyl group with hydrous ethanol, concentrating the extract, and purifying by column chromatography.

The composition for inhibiting reactive oxygen species production of the present invention may contain, as long as the effect thereof is not impaired, an optional component in addition to triterpenic acid having an acetyl group and/or a salt thereof. Examples of such optional components include bioactive components such as vitamins, minerals, hormones, proteins, amino acids, carbohydrates, polyunsaturated fatty acids, triglycerides containing such fatty acids as constituent fatty acids, and nutrients; and emulsifiers, tonicity agents (tonicity adjusting agents), buffers, solubilizers, preservatives, and stabilizers that are blended when formulating pharmaceutical agents.

Next, how to use the composition for inhibiting reactive oxygen species production of the present invention will now be described.

The composition for inhibiting reactive oxygen species production of the present invention is preferably used to prevent, ameliorate, or treat a condition or a disease resulting from oxidative stress.

The composition for inhibiting reactive oxygen species production of the present invention contains triterpenic acid having an acetyl group and/or a salt thereof as an active ingredient.

As described above, triterpenic acid having an acetyl group and/or a salt thereof can suppress NOX. Thus, reactive oxygen species production can be inhibited, and oxidative stress resulting from reactive oxygen species can be inhibited.

Accordingly, the composition for inhibiting reactive oxygen species production of the present invention is useful for preventing, ameliorating, or treating a condition or a disease resulting from oxidative stress.

Herein, the phrase “preventing a condition or a disease” refers to increasing the resistance of a subject to a condition or a disease, or preventing the onset of a condition or a disease. Also, the phrase “ameliorating or treating a condition or a disease” herein refers to recovering a subject from a condition or a disease, reducing the severity of a condition or a disease, or preventing the progress of a condition or a disease.

Examples of conditions or diseases resulting from oxidative stress include conditions or diseases such as nerve cell damage, brain inflammation, allergic diseases (such as atopic dermatitis, allergic rhinitis (hay fever), allergic conjunctivitis, allergic gastroenteritis, bronchial asthma, pediatric asthma, food allergies, drug allergies, and hives), Parkinson's disease, cerebral infarction, cataract, epilepsy, spinal cord injury, arteriosclerosis, retinopathy of prematurity, chronic renal dysfunction, renal impairment, pancreatitis, myocardial infarction, adult respiratory distress syndrome, emphysema, collagen diseases such as chronic rheumatoid arthritis, vasculitis, edema, diabetic complications, UV damage, altitude sickness, porphyria, burns, frostbite, contact dermatitis, multiple organ failure, disseminated intravascular coagulation (DIC), cancer, aging, fatigue, and chronic fatigue syndrome.

The composition for inhibiting reactive oxygen species production of the present invention is preferably used to prevent, ameliorate, or treat such conditions or diseases resulting from oxidative stress.

Examples of nerve cell damage include cerebral nerve cell damage, e.g., conditions or diseases such as forgetfulness, decline in cognitive function, decline in memory, decline in concentration, decline in attention, decline in judgment, decline in spatial cognition, decline in neural activity, decline in neurotransmission, decline in cognitive flexibility, decline in executive function, decline in information processing speed, depressive symptoms, dementia, and Alzheimer's disease. In particular, the composition for inhibiting reactive oxygen species production of the present invention is useful for at least one condition or disease selected from the group consisting of forgetfulness, decline in cognitive function, decline in memory, decline in judgment, dementia, and Alzheimer's disease as a condition or a disease resulting from oxidative stress. The composition for inhibiting reactive oxygen species production of the present invention is suitably used to prevent, ameliorate, or treat cerebral dysfunction resulting from cerebral nerve cell damage, and is more preferably used to prevent, ameliorate, or treat at least one condition or disease selected from the group consisting of forgetfulness, decline in cognitive function, decline in memory, decline in judgment, dementia, and Alzheimer's disease.

The composition for inhibiting reactive oxygen species production of the present invention is preferably used to inhibit or ameliorate a decline in brain function.

Examples of conditions or diseases resulting from a decline in brain function include conditions or diseases such as forgetfulness, decline in cognitive function, decline in memory, decline in concentration, decline in attention, decline in judgment, decline in spatial cognition, decline in neural activity, decline in neurotransmission, decline in cognitive flexibility, decline in executive function, decline in information processing speed, depressive symptoms, dementia, and Alzheimer's disease. In particular, the composition for inhibiting reactive oxygen species production of the present invention is useful for at least one condition or disease selected from the group consisting of forgetfulness, decline in cognitive function, decline in memory, decline in judgment, dementia, and Alzheimer's disease as a condition or a disease resulting from a decline in brain function.

Herein, the phrase “inhibiting or ameliorating a decline in brain function” refers to maintaining a brain function, inhibiting and/or preventing the progress of a decline in brain function, or ameliorating a decline in brain function.

The decline in brain function is preferably a decline in brain function associated with aging of a subject. As described above, NOX activity increases with age, and thus reactive oxygen species increases in a living body.

The composition for inhibiting reactive oxygen species production of the present invention can inhibit reactive oxygen species production, can inhibit oxidative stress resulting from reactive oxygen species, and is thus considered to be useful for inhibiting or ameliorating an age-related decline in brain function.

The composition for inhibiting reactive oxygen species production of the present invention may be an oral composition or an external composition.

The composition for inhibiting reactive oxygen species production of the present invention may also be used as a food or drink, an external agent, a drug, or a quasi drug.

In particular, the composition for inhibiting reactive oxygen species production of the present invention is preferably an oral composition, and is more preferably, for example, a food or drink, a drug (an oral drug), or a quasi drug (an oral quasi drug).

The method for producing the above food or drink, external agent, drug, or quasi drug is not limited, and the above triterpenic acid having an acetyl group and/or a salt thereof may be added thereto. The composition for inhibiting reactive oxygen species production of the present invention may be a raw material composition to be used as a raw material for such a food or drink, an external agent, a drug, or a quasi drug.

Herein, the term “food or drink” is a generic term for a solid, a fluid and a liquid, and a mixture thereof that can be orally taken. Examples of the food or drink include foods for specified health use, foods with functional claims, nutritionally functional foods, nutritional supplementary foods, health foods, functional foods, foods for infants, and foods for elders.

The food for specified health use means a food for which permission under Article 26, Paragraph 1 of the Health Promotion Act or approval under Article 29, Paragraph 1 of the same Act has been obtained to indicate to persons who consume the food for specified health purposes for their daily diet that they can expect to fulfill the health purposes by consuming the food.

The food with functional claims refers to a food that, on the responsibility of a business operator, indicates nutritional function based on scientific evidence under Article 2, Paragraph 1, Item 10 of the Food Labeling Standards, and for which information concerning the basis of safety and functionality is submitted to the Commissioner of the Consumer Affairs Agency before commercial distribution.

The nutritionally functional food refers to a food that can be used to replenish and supplement nutrients that are required each day and that are likely insufficient from daily diet.

The form of the food or drink is not limited, and the food or drink may have a variety of forms. Examples include dietary supplements, beverages (alcoholic beverages and non-alcoholic beverages), and confections. In the case of a dietary supplement, the food or drink can be in a variety of preparation forms, e.g., oral solid preparations such as tablets, coated tablets, fine granules, granules, powders, pills, capsules (including soft capsules and hard capsules), dry syrups, and chewables; and oral liquid preparations such as internal liquid agents and syrups. In the case of a tablet, a pill, and a granule, the food or drink can be in a dosage form optionally provided with a commonly used coating, such as a sugar-coated tablet, a gelatin-coated agent, an enteric-coated agent, or a film-coated agent, and the tablet may be a multilayer tablet such as a double-layer tablet.

When using the composition for inhibiting reactive oxygen species production of the present invention as a food or drink, the food or drink may contain sweeteners such as stevia extracts, acesulfame potassium, and sucralose, carbohydrates such as sucrose, organic acids such as malic acid and citric anhydride, dietary fibers such as indigestible dextrin, flavors, and colorants.

When used as an external agent, the composition for inhibiting reactive oxygen species production of the present invention is preferably used as a skin external composition.

When used as a skin external composition, the composition for inhibiting reactive oxygen species production of the present invention can be prepared as skin care cosmetics such as tapes, ointments, facial washes (facial cleansers), cosmetic water, masks, massage creams, milky lotions, emulsions, creams, and essences (beauty serums); and body care cosmetics such as soaps, liquid cleaning agents, bath additives, sunblocks, tanning oils, deodorant sprays, body lotions, body creams, and hand creams.

The cosmetics may further contain carriers and excipients that can be used in cosmetics.

When using the composition for inhibiting reactive oxygen species production of the present invention as a drug or a quasi drug, the route of administration of the drug or the quasi drug may be oral or parenteral, and is preferably oral.

Examples of parenteral administration include transdermal, topical, transnasal, sublingual, transpulmonary, enteral, transmucosal, and injection administrations.

Dosage forms of oral preparations include liquids, tablets, powders, pills, fine granules, granules, sugar-coated tablets, capsules, suspensions, emulsions, syrups, extracts, emulsions, microcapsules, lozenges, candies, buccal agents, and chewables.

Dosage forms of parenteral preparations include aerosols, injections, drips, inhalants, infusions, suppositories, transdermal absorbents, nasal drops, eye drops, cataplasms, adhesive skin poultices, creams, gels, and lotions.

When using the composition for inhibiting reactive oxygen species production of the present invention as a drug or a quasi drug, the composition for inhibiting reactive oxygen species production of the present invention may be used alone or may be used in combination with other drugs or quasi drugs.

Moreover, when using the composition for inhibiting reactive oxygen species production of the present invention as a drug or a quasi drug, the drug or the quasi drug may contain any one or more additives selected from the group consisting of pharmaceutically acceptable carriers, excipients, buffers, binders, disintegrants, lubricants, antioxidants, and colorants.

For example, the composition for inhibiting reactive oxygen species production of the present invention may be used in the form of, but is not limited to, an agent. The agent may be directly used as a composition for inhibiting reactive oxygen species production, and may be used as a composition for inhibiting reactive oxygen species production containing the agent.

When the composition for inhibiting reactive oxygen species production of the present invention is a raw material composition, the content of triterpenic acid having an acetyl group and/or a salt thereof is not limited and may be set according to the form of the composition. For example, the content is preferably 0.01% by weight or more, more preferably 0.1 to 99% by weight, even more preferably 1 to 70% by weight.

When two or more kinds of triterpenic acid having an acetyl group and/or a salt thereof are contained, the above content is the total content thereof.

When orally taking the composition for inhibiting reactive oxygen species production of the present invention, the daily intake of triterpenic acid having an acetyl group and/or a salt thereof is preferably 0.01 to 200 mg, more preferably 0.1 to 100 mg, and, in one embodiment, even more preferably 8 to 17 mg.

When two or more kinds of triterpenic acid having an acetyl group and/or a salt thereof are taken, the above intake is the total intake thereof.

When using the composition for inhibiting reactive oxygen species production of the present invention as an external agent such as a skin external composition, the daily dose of triterpenic acid having an acetyl group and/or a salt thereof is preferably 0.01 to 200 mg, more preferably 0.1 to 100 mg. This dose may be used in one portion or may be used in divided portions.

When two or more kinds of triterpenic acid having an acetyl group and/or a salt thereof are used, the above dose is the total dose thereof.

By continuously taking (administering) the composition for inhibiting reactive oxygen species production of the present invention, the effect of inhibiting reactive oxygen species production is expected to be increased. In a preferable embodiment, the composition for inhibiting reactive oxygen species production of the present invention is continuously taken. In one embodiment of the present invention, the composition for inhibiting reactive oxygen species production is preferably taken continuously for at least two weeks or longer. More preferably, the composition is continuously taken for 12 weeks or longer.

The subject that takes or is administered with the composition for inhibiting reactive oxygen species production of the present invention is preferably a human or a non-human mammal.

The subject that takes or is administered with the composition for inhibiting reactive oxygen species production of the present invention may be a subject that wishes or needs inhibition of reactive oxygen species.

In one embodiment, it may be a subject that wishes or needs prevention, amelioration, or treatment of the above-described condition or disease resulting from oxidative stress.

The composition for inhibiting reactive oxygen species production of the present invention may be labeled as being intended for preventing, ameliorating, or treating a condition or a disease resulting from oxidative stress or a decline in brain function. In one embodiment, the composition may be labeled as being intended for preventing, ameliorating, or treating at least one condition or disease selected from the group consisting of, for example, forgetfulness, decline in cognitive function, decline in memory, decline in judgment, dementia, and Alzheimer's disease.

The present invention also encompasses the following method and use.

That is to say, a method for inhibiting reactive oxygen species production including administering triterpenic acid having an acetyl group and/or a salt thereof to a subject is also one aspect of the present invention.

Also, the use of triterpenic acid having an acetyl group and/or a salt thereof for inhibiting reactive oxygen species production is one aspect of the present invention.

Concerning triterpenic acid having an acetyl group and/or a salt thereof in the method and use, the intake (dosage), dosing subject, and preferred embodiments thereof are the same as those described above in connection with the composition for inhibiting reactive oxygen species production. The method and use may be therapeutic, and may be non-therapeutic. The term “non-therapeutic” refers to a concept that excludes a medical procedure, i.e., surgery, treatment, or diagnosis.

The present invention also encompasses the following composition for inhibiting or ameliorating a decline in brain function, method for inhibiting or ameliorating a decline in brain function, and use of triterpenic acid having an acetyl group and/or a salt thereof for inhibiting or ameliorating a decline in brain function.

That is to say, a composition for inhibiting or ameliorating a decline in brain function, which contains triterpenic acid having an acetyl group and/or a salt thereof as an active ingredient, is also one aspect of the present invention.

Concerning triterpenic acid having an acetyl group and/or a salt thereof in the composition for inhibiting or ameliorating a decline in brain function, the same triterpenic acid having an acetyl group and/or a salt thereof as used in the composition for inhibiting reactive oxygen species production of the present invention can be used. In particular, it is preferably at least one selected from the group consisting of 3-acetylursolic acid, 3-acetyloleanolic acid, and salts thereof, in which an acetyl group is bonded to the hydroxyl group at 3-position.

The disease or the condition resulting from a decline in brain function is preferably at least one condition or disease selected from the group consisting of forgetfulness, decline in cognitive function, decline in memory, decline in concentration, decline in attention, decline in judgment, decline in spatial cognition, decline in neural activity, decline in neurotransmission, decline in cognitive flexibility, decline in executive function, decline in information processing speed, depressive symptoms, dementia, and Alzheimer's disease. In particular, the composition for inhibiting or ameliorating a decline in brain function of the present invention is useful for at least one condition or disease selected from the group consisting of forgetfulness, decline in cognitive function, decline in memory, decline in judgment, dementia, and Alzheimer's disease, and, inter alia, useful for inhibiting or ameliorating a decline in cognitive function, in particular a decline in memory function.

The composition for inhibiting or ameliorating a decline in brain function of the present invention may contain, as long as the effect thereof is not decreased, an optional component in addition to triterpenic acid having an acetyl group and/or a salt thereof. Such optional components may be the same as those that may be contained in the composition for inhibiting reactive oxygen species production of the present invention.

The composition for inhibiting or ameliorating a decline in brain function of the present invention, as with the composition for inhibiting reactive oxygen species production of the present invention, may be an oral composition or an external composition, and may be used as a food or drink, an external agent, a drug, or a quasi drug.

When using the composition for inhibiting or ameliorating a decline in brain function of the present invention as the above food or drink, external agent, drug, or quasi-drug, the production method therefor is not limited, and can be the same as the production method for the composition for inhibiting reactive oxygen species.

For example, the composition for inhibiting or ameliorating a decline in brain function of the present invention may be used in the form of, but is not limited to, an agent. The agent may be directly used as a composition for inhibiting or ameliorating a decline in brain function, and may be used as a composition for inhibiting or ameliorating a decline in brain function containing the agent.

When the composition for inhibiting or ameliorating a decline in brain function of the present invention is a raw material composition, the content of triterpenic acid having an acetyl group and/or a salt thereof may be the same as that in the raw material composition for the composition for inhibiting reactive oxygen species production of the present invention.

When orally taking the composition for inhibiting or ameliorating a decline in brain function of the present invention or using it as an external agent, the daily dosage, the dosing (administration) method, the dosing (administration) subject of triterpenic acid having an acetyl group and/or a salt thereof can be the same as those concerning the composition for inhibiting reactive oxygen species production.

Also, the composition for inhibiting or ameliorating a decline in brain function can be used to inhibit or ameliorate an age-related decline in brain function.

The composition for inhibiting or ameliorating a decline in brain function of the present invention may be labeled as being intended for preventing, ameliorating, or treating a condition or a disease resulting from a decline in brain function. In one embodiment, the composition may be labeled as being intended for preventing, ameliorating, or treating at least one condition or disease selected from the group consisting of, for example, forgetfulness, decline in cognitive function, decline in memory, decline in judgment, dementia, and Alzheimer's disease.

A method for inhibiting or ameliorating a decline in brain function including administering triterpenic acid having an acetyl group and/or a salt thereof to a subject is also one aspect of the present invention. In this case, the decline in brain function may be an age-related decline in brain function.

Also, the use of triterpenic acid having an acetyl group and/or a salt thereof for inhibiting or ameliorating a decline in brain function is one aspect of the present invention. In this case, the decline in brain function may be an age-related decline in brain function.

Concerning triterpenic acid having an acetyl group and/or a salt thereof in the method and use, the intake (dosage), dosing subject, and preferred embodiments thereof are the same as those described above in connection with the composition for inhibiting reactive oxygen species production. The method and use may be therapeutic, and may be non-therapeutic. The term “non-therapeutic” refers to a concept that excludes a medical procedure, i.e., surgery, treatment, or diagnosis.

### EXAMPLES

The composition for inhibiting reactive oxygen species production of the present invention will now be described in more detail by way of Examples below, but the scope of the present invention is not limited by these Examples.

### Example 1: Measurement of NOX Inhibitory Activity Using Standard Product

### (1) Preparation of Differentiated HL-60 Cells

It is known that undifferentiated human myeloid leukemia HL-60 cells have an ability to proliferate, but differentiate into mature granulocytes by adding DMSO (dimethyl sulfoxide), retinoic acid, or the like and lose their proliferative ability. Mature HL-60 cells express NOX that serves as an indicator of differentiation. NOX can be used as an indicator for evaluating NOX inhibitory activity.

HL-60 cells were differentiated into NOX-expressing granulocytes by the following method.

First, undifferentiated HL-60 cells cultured in a 10% FBS-containing RPMI 1640 medium were suspended in a 10% FBS-containing RPMI 1640 medium containing 1% DMSO so as to have 5×10⁵ cells/mL. Then, the suspension was dispensed into cell culture dishes (100 mm) so as to be 15 mL each, and the cells were cultured in a CO₂ incubator (37° C.) for 3 days.

Then, each cell culture dish was added with 10 mL of a 10% FBS-containing RPMI 1640 medium containing 1% DMSO, and the cells were further cultured for 3 days.

Through the above steps, HL-60 cells were differentiated into NOX-expressing granulocytes.

The differentiated HL-60 cells were used to measure NOX inhibitory activity as will be described below.

### (2) Preparation of Measurement Reagent

Commercially available standard products of 3-acetylursolic acid, 3-acetyloleanolic acid, ursolic acid, and oleanolic acid were used.

### (2-1) Preparation of Group of 3-Acetylursolic Acid Test Sample Solutions

3-Acetylursolic acid was dissolved in DMSO such that the concentration of 3-acetylursolic acid was 2.5 mg/mL, a series of two-fold dilutions were prepared therefrom using DMSO, and the dilutions were each further diluted with a D-MEM medium to prepare a group of 3-acetylursolic acid test sample solutions having a final concentration of 25, 12.5, 6.3, 3.1, and 1.6 μM.

### (2-2) Preparation of Group of 3-Acetyloleanolic Acid Test Sample Solutions

3-Acetyloleanolic acid was dissolved in DMSO such that the concentration of 3-acetyloleanolic acid was 2.5 mg/mL, a series of two-fold dilutions were prepared therefrom using DMSO, and the dilutions were each further diluted with a D-MEM medium to prepare a group of 3-acetyloleanolic acid test sample solutions having a final concentration of 25, 12.5, 6.3, 3.1, and 1.6 μM.

### (2-3) Preparation of Group of Ursolic Acid Test Sample Solutions

Ursolic acid was dissolved in DMSO such that the concentration of ursolic acid was 5 mg/mL, a series of two-fold dilutions were prepared therefrom using DMSO, and the dilutions were each further diluted with a D-MEM medium to prepare a group of ursolic acid test sample solutions having a final concentration of 50, 25, 12.5, 6.3, 3.1, and 1.6 μM.

### (2-4) Preparation of Group of Oleanolic Acid Test Sample Solutions

Oleanolic acid was dissolved in DMSO such that the concentration of oleanolic acid was 5 mg/mL, a series of two-fold dilutions were prepared therefrom using DMSO, and the dilutions were each further diluted with a D-MEM medium to prepare a group of oleanolic acid test sample solutions having a final concentration of 50, 25, 12.5, 6.3, 3.1, and 1.6 μM.

### (2-5) Preparation of WST-1 Solution

WST-1 (2-(4-iodophenyl)-3-(4-nitrophenyl)-5-(2,4-disulfophenyl)-2H-tetrazolium, monosodium salt) was dissolved in a D-MEM medium so as to be 0.8 mg/mL, and thus a WST-1 solution was prepared. WST-1 reacts with superoxide and produces yellow formazan. Yellow formazan absorbs a wavelength of 450 nm, and, therefore, the amount of yellow formazan can be measured by measuring absorbance at a wavelength of 450 nm. Also, the amount of yellow formazan produced is proportional to the amount of superoxide during the reaction, and, therefore, the amount of superoxide can be measured by measuring the amount of yellow formazan produced.

### (2-6) Preparation of PMA Solution

PMA (phorbol 12-myristate 13-acetate) was dissolved in a D-MEM medium so as to be 4 μM, and thus a PMA solution was prepared.

PMA is known to function to activate NOX and causes superoxide production.

### (3) Measurement of NOX Inhibitory Activity

The differentiated HL-60 cells were collected by centrifugation, and suspended in a D-MEM medium containing neither FBS nor phenol red so as to be 5×10⁶ cells/mL.

The cell suspension was added into a 96-well microplate so as to be 25 μL per well.

Next, 25 μL of the WST-1 solution prepared as described above was added to the wells.

Moreover, 25 μL each of the prepared test sample solutions of 3-acetylursolic acid, 3-acetyloleanolic acid, ursolic acid, oleanolic acid, or a DMEM medium was added to the wells.

After adding the solutions, the cell suspensions were stirred.

Thereafter, 25 μL of the prepared PMA solution was added (a final PMA concentration in the medium being 1 μM) to activate NOX and carry out a reaction at 37° C. for 60 minutes.

Then, by measuring absorbance at a wavelength of 450 nm, the amount of yellow formazan produced and thus the amount of superoxide produced by NOX were measured.

In this reaction system, NOX is not activated unless PMA is added to the medium. Moreover, when VAS2870 that is a known NOX inhibitor was evaluated as a positive control, the IC₅₀ was 8 μM. From these, it was verified that this evaluation system is suitable for evaluating NOX inhibitory activity.

Based on the obtained measurement results, IC₅₀ (4M) of 3-acetylursolic acid, 3-acetyloleanolic acid, ursolic acid, and oleanolic acid with respect to NOX was determined. The inhibitory activity ratio of 3-acetylursolic acid to ursolic acid and the inhibitory activity ratio of 3-acetyloleanolic acid to oleanolic acid were calculated using formula (i) below:

Inhibitory activity ratio=(IC₅₀ of triterpenic acid having acetyl group)/(IC₅₀ of triterpenic acid without acetyl group) (i)

The results are shown in Table 1.

TABLE 1

| | | Inhibitory |
|---|---|---|
| Compound | IC₅₀ (μM) | activity ratio |
| 3-Acetylursolic acid | 6 | 7.3 times |
| Ursolic acid | 44 | — |
| 3-Acetyloleanolic acid | 10 | 4.4 times |
| Oleanolic acid | 44 | — |

As shown in Table 1, it was found that 3-acetylursolic acid and 3-acetyloleanolic acid have NOX inhibitory activity comparable to that of VAS2870 (described above) known as a NOX inhibitor and, moreover, have higher NOX inhibitory activity than ursolic acid and oleanolic acid that do not have an acetyl group. Accordingly, it was demonstrated that 3-acetylursolic acid and 3-acetyloleanolic acid are useful as active ingredients in a composition for inhibiting reactive oxygen species production. Also, it has become clear that acetylated triterpenes can increase NOX inhibitory activity.

### Example 2: Measurement of NOX Inhibitory Activity Using Plant Extract

### (1) Preparation of Aronia Extract

Pomace of Aronia melanocarpa was freeze-dried and then ground. Ground sample was extracted with hydrous ethanol, then filtered, and concentrated under reduced pressure to give an extract powder. This extract powder was dissolved in water and extracted with ethyl acetate five times. The ethyl acetate layer was subjected to Diaion HP-20 and eluted with different concentrations of hydrous ethanol. The ethanol eluate was subjected to Silica Gel 60N, and eluted with different concentrations of hexane/ethyl acetate mixtures. The eluates were sequentially verified by thin-layer chromatography, and eluates having the same spot pattern were combined into a single fraction. Thereby, multiple fractions were obtained.

### (2) Qualitative and Quantitative Analysis of Triterpenic Acids

Standard products of 3-acetylursolic acid, 3-acetyloleanolic acid, ursolic acid, and oleanolic acid were provided. Then, the standard products and the above fractions were analyzed by the method disclosed in a patent literature (JP 5466842 B) under the following conditions.

### (2-1) HPLC Method

The above standard products and the above fractions were subjected to a high performance liquid chromatography analysis under the following conditions.

Column: Develosil C-30-UG5 (ϕ4.5 μm×250 mm) manufactured by Nomura Chemical Co., Ltd.

Mobile phase solvent: 90% acetonitrile, 0.05% trifluoroacetic acid

Flow rate: 0.5 mL/min

Detection: UV (210 nm), ELSD

The retention time of a peak in the chromatogram of each fraction and the retention time of each standard product were compared to determine the compounds. The retention time of each compound under the above analysis method was oleanolic acid, 11.42 min; ursolic acid, 11.94 min; 3-acetyloleanolic acid, 16.77 min; and 3-acetylursolic acid, 17.53 min.

Among the fractions, a fraction containing ursolic acid and oleanolic acid was referred to as a fraction of triterpenic acid without an acetyl group (fraction 1), and a fraction containing 3-acetylursolic acid and 3-acetyloleanolic acid was referred to as a fraction of triterpenic acid having an acetyl group (fraction 2).

### (2-2) Method for Calculating Purity of Triterpenes

Concerning fraction 1 and fraction 2, the contents of triterpenic acids having an acetyl group or triterpenic acid without an acetyl group were calculated according to the following mathematical formulae (ii) and (iii) based on the ELSD chromatogram obtained by the above analysis. The results are shown in Table 2.

Fraction 1: Content(%) of triterpenic acid without acetyl group=[(Peak area of oleanolic acid+Peak area of ursolic acid)/Total peak area]×100 (ii)

Fraction 2: Content(%) of triterpenic acid having acetyl group=[(Peak area of 3-acetyloleanolic acid+Peak area of 3-acetylursolic acid)/Total peak area]×100 (iii)

### (3) Measurement of NOX Inhibitory Activity

### (3-1) Preparation of Group of Test Samples of Fraction 1

Fraction 1 was dissolved in DMSO such that the concentration was 10 mg/mL, a series of two-fold dilutions were prepared therefrom using DMSO, and the dilutions were each further diluted with a D-MEM medium to prepare a group of test sample solutions of fraction 1 having a final concentration of triterpenic acid without an acetyl group of 76, 38, 19, and 9.5 μg/mL.

### (3-2) Preparation of Group of Test Samples of Fraction 2

Fraction 2 was dissolved in DMSO such that the concentration was 10 mg/mL, a series of two-fold dilutions were prepared therefrom using DMSO, and the dilutions were each further diluted with a D-MEM medium to prepare a group of test sample solutions of fraction 2 having a final concentration of triterpenic acid having an acetyl group of 96, 48, 24, 12, 6, 3, 1.5, and 0.75 μg/mL.

### (3-3) Preparation of Other Reagents

A control solution, a WST-1 solution, and a PMA solution were prepared in the same manner as in Example 1.

### (4) Measurement of NOX Inhibitory Activity

The NOX inhibitory activities of fraction 1 and fraction 2 were measured in the same manner as in Example 1 except that the group of test samples of fraction 1 and the group of test samples of fraction 2 were used in place of the group of test sample solutions of each standard product.

Based on the obtained measurement results, the IC₅₀ (μg/mL) of fraction 1 and fraction 2 was determined.

The IC₅₀ of fraction 1 was 80 μg/mL, and the IC₅₀ of fraction 2 was 4 μg/mL.

Moreover, the inhibitory activity ratio of triterpenic acid having an acetyl group in fraction 2 to triterpenic acid without an acetyl group in fraction 1 was calculated using formula (i).

The results are shown in Table 2.

TABLE 2

| | Content in | Content in | | Inhibitory |
|---|---|---|---|---|
| | fraction 1 | fraction 2 | IC₅₀ | activity |
| Component | (%) | (%) | (μg/mL) | ratio |
| Triterpenic acid without | 76.2 | 0.0 | 61 | — |
| acetyl group | | | | |
| Triterpenic acid having | 0.0 | 96.2 | 3.8 | 16 times |
| acetyl group | | | | |

As shown in Table 2, it was found that triterpenic acid having an acetyl group in fraction 2 had higher NOX inhibitory activity than triterpenic acid without an acetyl group in fraction 1.

### Reference Example 1: Evaluation of Brain Function Using Senescence Accelerated Model

The effect of inhibiting a decline in brain function of the Aronia extract was evaluated using the extract powder obtained in Example 2. The Senescent Accelerated Model Prone 10 (SAMP10) mouse, which is one of the senescence accelerated models, is known to show age-related decline in brain function at an early stage, and can be used to evaluate effects on an age-related decline in brain function. Accordingly, this animal was used to investigate the brain function ameliorating effect of the Aronia extract. A series of animal experiments were carried out based on a plan that complies with the animal welfare management law and other relevant laws and that is approved by the head of an organization after being reviewed by an institutional animal experiment committee.

As for a young (young control) group, SAMP10 mice at 8 weeks old were purchased, acclimated for 7 days, and received AIN-93M (manufactured by CLEA Japan Inc.) as a base food for 12 weeks, and then the brain function was evaluated at 4 months old (N=15). As for an elderly group, mice at 14 weeks old were purchased, acclimated until 8 months old, randomly divided into 2 groups, received AIN-93M or AIN-93M containing 0.02% Aronia extract for 12 weeks, and then the brain function was evaluated at 11 months old (an elderly control group (N=26), an elderly Aronia received group (N=18)). The food was changed once a week, and the food intake and the body weight were measured once a week.

### (1) Brain Function Evaluation (Passive Avoidance Test)

A brain function was evaluated using a passive avoidance test (a step-through test). It is known that the passive avoidance response is a learned behavior for escaping unpleasant stimuli, and the passive avoidance test is a system that enables to evaluate brain functions such as memory.

### (1-1): Acquisition Trial

Mice were placed in a light room (9 cm width, 11.5 cm depth, 15 cm height) of a step-through passive avoidance response tester (manufactured by Muromachi Kikai Co., Ltd.), then a door to a dark room (14 cm width, 17.5 cm depth, 15 cm height) was opened 30 seconds later, and the time until the mice entered the dark room (the light room latency) was measured. After measurement, the mice in the dark room received a weak electrical current of 0.20 mA for 1 second, and then accommodated in the original cage.

### (1-2): Retention Trial

A retention trial was performed the day after the acquisition trial. The same operation as the acquisition trial was performed on the mice, and the latency in light room was measured. However, no electrical current stimulation was given to the mice that entered the dark room. The latency was measured up to 300 seconds, and evaluations were made such that the longer the latency of the retention trial is, the higher the memory function is.

### (2) Results

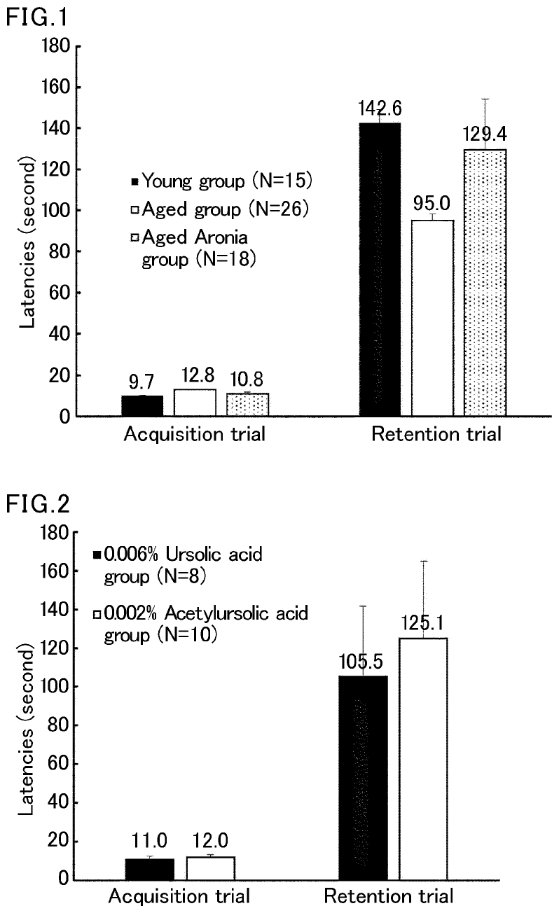

There was no difference between the young control group, the elderly control group, and the elderly Aronia received group in body weight and food consumption. FIG. 1 shows the results of evaluating the latency in light room of the acquisition trial and the retention trial. There was no difference between groups in the latency of the acquisition trial. For the retention trial, a significantly shorter the latency was observed in the aged control group than the young control group. This indicates that it was possible to evaluate an age-related decline in brain function in this test. In the retention trial, a significant recovery of light room latency was observed in the elderly Aronia extract received group compared with the elderly control group. From these results, it was found that the Aronia extract inhibits an age-related decline in brain function.

### Example 3: Comparison Between Ursolic Acid and Acetylursolic Acid Using Senescence Accelerated Model

The brain function ameliorating effects of ursolic acid and acetylursolic acid contained in Aronia were compared in terms of the recovery of light room latency. SAMP10 mice at 14 weeks old were purchased, acclimated until 8 months old, then randomly divided into two groups, and received AIN-93M containing 0.006% ursolic acid or AIN-93M containing 0.002% acetylursolic acid for 12 weeks. When the mice reached 11 months old, a brain function was evaluated according to the method of Reference Example 1 (0.006% ursolic acid received group (N=8), and 0.002% acetylursolic acid received group (N=10)).

### (1) Preparation of Evaluation Sample

A commercially available ursolic acid reagent was purchased and used (95% or higher purity). 3-Acetylursolic acid was synthesized by a reaction in which an acetyl group was added to ursolic acid, and purified by silica gel. When the resulting white powder was analyzed by the method described in (2-1) Conditions for analyzing triterpenes above, a peak was confirmed at the same retention time as the 3-acetylursolic acid standard product, and no other main peaks were detected. 3-Acetylursolic acid thus obtained was used as an evaluation sample.

### (2) Results

There was no difference between the ursolic acid received group and the acetylursolic acid received group in body weight and food intake. FIG. 2 shows the results of evaluating the latency in light room of the acquisition trial and the retention trial. There was no difference between groups in the latency of the acquisition trial. For the retention trial, acetylursolic acid in a lower dose showed a stronger effect on ameliorating brain function than ursolic acid that is known to ameliorate brain function. From this result, it was found that ursolic acid and acetylursolic acid act to inhibit an age-related decline in brain function, and triterpenic acid having an acetyl group has a stronger effect of inhibiting an age-related decline in brain function than triterpenic acid that does not have an acetyl group.

### INDUSTRIAL APPLICABILITY

The composition for inhibiting reactive oxygen species production of the present invention is useful in, for example, the food or drink field and the pharmaceutical field.

1. A composition for inhibiting reactive oxygen species production, comprising triterpenic acid having an acetyl group and/or a salt thereof as an active ingredient.

2. The composition for inhibiting reactive oxygen species production according to claim 1,
wherein the composition inhibits reactive oxygen species production by suppressing NADPH oxidase (NOX).

3. The composition for inhibiting reactive oxygen species production according to claim 1,
wherein the triterpenic acid having an acetyl group and/or the salt thereof is at least one selected from the group consisting of 3-acetylursolic acid, 3-acetyloleanolic acid, and salts thereof.

4. The composition for inhibiting reactive oxygen species production according to claim 1, for use in preventing, ameliorating, or treating a condition or a disease resulting from oxidative stress.

5. The composition for inhibiting reactive oxygen species production according to claim 1, for use in inhibiting or ameliorating a decline in brain function.

6. The composition for inhibiting reactive oxygen species production according to claim 5,
wherein the decline in brain function is an age-related decline in brain function.

7. composition for inhibiting reactive oxygen species production according to claim 4,
wherein the condition or the disease resulting from the oxidative stress or the decline in brain function is at least one condition or disease selected from the group consisting of forgetfulness, decline in cognitive function, decline in memory, decline in judgment, dementia, and Alzheimer's disease.

8. The composition for inhibiting reactive oxygen species production according to claim 1,
wherein the composition is labeled as being intended for preventing, ameliorating, or treating a condition or a disease resulting from oxidative stress or a decline in brain function.

9. The composition for inhibiting reactive oxygen species production according to claim 1,
wherein the composition is an oral composition.

10. The composition for inhibiting reactive oxygen species production according to claim 9,
wherein the oral composition is a food or drink, a drug, or a quasi drug.

11. A method for inhibiting reactive oxygen species production, comprising administering triterpenic acid having an acetyl group and/or a salt thereof to a subject.

12. Use of triterpenic acid having an acetyl group and/or a salt thereof for inhibiting reactive oxygen species production.

13. A composition for inhibiting or ameliorating a decline in brain function, comprising triterpenic acid having an acetyl group and/or a salt thereof as an active ingredient.

14. The composition for inhibiting or ameliorating a decline in brain function according to claim 13, for use in inhibiting or ameliorating an age-related decline in brain function.

15. The composition for inhibiting or ameliorating a decline in brain function according to claim 13,
wherein the triterpenic acid having an acetyl group and/or the salt thereof is at least one selected from the group consisting of 3-acetylursolic acid, 3-acetyloleanolic acid, and salts thereof.

16. The composition for inhibiting or ameliorating a decline in brain function according to claim 13,
wherein the disease or the condition resulting from the decline in brain function is at least one condition or disease selected from the group consisting of forgetfulness, decline in cognitive function, decline in memory, decline in judgment, dementia, and Alzheimer's disease.

17. The composition for inhibiting or ameliorating a decline in brain function according to claim 13,
wherein the composition is labeled as being intended for preventing, ameliorating, or treating a condition or a disease resulting from a decline in brain function.

18. The composition for inhibiting or ameliorating a decline in brain function according to claim 13, wherein the composition is an oral composition.

19. The composition for inhibiting or ameliorating a decline in brain function according to claim 18,
wherein the oral composition is a food or drink, a drug, or a quasi drug.

20. A method for inhibiting or ameliorating a decline in brain function, comprising administering triterpenic acid having an acetyl group and/or a salt thereof to a subject.

21. Use of triterpenic acid having an acetyl group and/or a salt thereof for inhibiting or ameliorating a decline in brain function.
